(19) United States
(12) Patent Application Publication
(10) Pub. No.: US 2005/0026931 A1
(43) Pub. Date: Feb. 3, 2005

(54) Substituted n-phenyl 2-hydroxy-2-methyl-3,3,3-trifluropropanamide derivatives which elevate pyruvate dehydrogenase activity

(76) Inventor: Roger John Butlin, Macclesfield (GB)

Correspondence Address: MORGAN LEWIS & BOCKIUS LLP, 1111 PENNSYLVANIA AVENUE NW, WASHINGTON, DC, 20004, US

(21) Appl. No.: 10/487,261

(22) PCT Filed: Aug. 21, 2002
(86) PCT No.: PCT/GB02/03867

(30) Foreign Application Priority Data
Aug. 23, 2001 (GB) 01204718

(51) Int. Cl. A61K 31/495; C07D 241/04

(52) U.S. Cl. 514/255.03; 544/392

(57) ABSTRACT

Compounds of formula (I) wherein R is methyl or mesyl; and pharmaceutically acceptable salts and in vivo hydrolysable esters thereof are described. Also described are processes for their preparation, pharmaceutical compositions containing it and their use in producing an elevation of PDH activity in a warm-blooded animal.

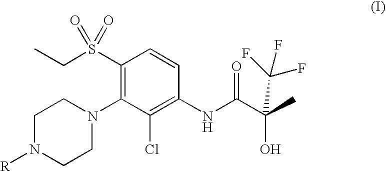

The present invention relates to compounds which elevate pyruvate dehydrogenase (PDH) activity, processes for their preparation, pharmaceutical compositions containing them as the active ingredient, methods for the treatment of disease states associated with reduced PDH activity, to their use as medicaments and to their use in the manufacture of medicaments for use in the elevation of PDH activity in warm-blooded animals such as humans. In particular this invention relates to compounds useful for the treatment of diabetes mellitus, peripheral vascular disease and myocardial ischaemia in warm-blooded animals such as humans, more particularly to the use of these compounds in the manufacture of medicaments for use in the treatment of diabetes mellitus in warm-blooded animals such as humans.

Within tissues adenosine triphosphate (ATP) provides the energy for synthesis of complex molecules and, in muscle, for contraction. ATP is generated from the breakdown of energy-rich substrates such as glucose or long chain free fatty acids. In oxidative tissues such as muscle the majority of the ATP is generated from acetyl CoA which enters the citric acid cycle, thus the supply of acetyl CoA is a critical determinant of ATP production in oxidative tissues. Acetyl CoA is produced either by β-oxidation of fatty acids or as a result of glucose metabolism by the glycolytic pathway. The key regulatory enzyme in controlling the rate of acetyl CoA formation from glucose is PDH which catalyses the oxidation of pyruvate to acetyl CoA and carbon dioxide with concomitant reduction of nicotinamide adenine dinucleotide (NAD) to NADH.

In disease states such as both non-insulin dependent (Type 2) and insulin-dependent (Type 1) diabetes mellitus, oxidation of lipids is increased with a concomitant reduction in utilisation of glucose, which contributes to the hyperglycaemia Reduced glucose utilisation in both Type 1 and Type 2 diabetes is associated with a reduction in PDH activity. In addition, a further consequence of reduced PDH activity may be that an increase in pyravate concentration results in increased availability of lactate as a substrate for hepatic gluconeogenesis. It is reasonable to expect that increasing the activity of PDH could increase the rate of glucose oxidation and hence overall glucose utilisation, in addition to reducing hepatic glucose output. Another factor contributing to diabetes mellitus is impaired insulin secretion, which has been shown to be associated with reduced PDH activity in pancreatic β-cells (in a rodent genetic model of diabetes mellitus Zhou et al. (1996) Diabetes 45: 580-586).

Oxidation of glucose is capable of yielding more ATP per mole of oxygen than is oxidation of fatty acids. In conditions where energy demand may exceed energy supply, such as myocardial ischaemia, intermittent claudication, cerebral ischaemia and reperfusion, (Zaidan et al., 1998; J. Neurochem. 70: 233-241), shifting the balance of substrate utilisation in favour of glucose metabolism by elevating PDH activity may be expected to improve the ability to maintain ATP levels and hence function.

An agent which is capable of elevating PDH activity may also be expected to be of benefit in treating conditions where an excess of circulating lactic acid is manifest such as in certain cases of sepsis.

The agent dichloroacetic acid (DCA) which increases the activity of PDH after acute administration in animals, (Vary et al., 1988; Circ. Shock, 24: 3-18), has been shown to have the predicted effects in reducing glycaemia, (Stacpoole et al., 1978; N. Engl. J. Med. 298: 526-530), and as a therapy for myocardial ischaemia Bersin and Stacpoole 1997; American Heart Journal, 134: 841-855) and lactic acidaemia, (Stacpoole et al., 1983; N. Engl. J. Med. 309: 390-396).

PDH is an intramitochondrial multienzyme complex consisting of multiple copies of several subunits including three enzyme activities E1, E2 and E3, required for the completion of the conversion of pyruvate to acetyl CoA (Patel and Roche 1990; FASEB J., 4: 3224-3233). E1 catalyses the irreversible loss of CO₂ from pyruvate; E2 forms acetyl CoA and E3 reduces NAD to NADH. Two additional enzyme activities are associated with the complex: a specific kinase which is capable of phosphorylating E1 at three serine residues and a loosely-associated specific phosphatase which reverses the phosphorylation. Phosphorylation of a single one of the three serine residues renders the E1 inactive. The proportion of the PDH in its active (dephosphorylated) state is determined by a balance between the activity of the kinase and phosphatase. The activity of the kinase may be regulated in vivo by the relative concentrations of metabolic substrates such as NAD/NADH, CoA/acetylCoA and adenosine diphosphate (ADP)/ATP as well as by the availability of pyruvate itself.

A compound that elevates PDH activity may potentially have value in the treatment of disease states associated with disorders of glucose utilisation such as diabetes mellitus, obesity, (Curto et al., 1997; Int. J. Obes. 21: 1137-1142), and lactic acidaemia. Additionally such a compound may be expected to have utility in diseases where supply of energy-rich substrates to tissues is limiting such as peripheral vascular disease, (including intermittent claudication), cardiac failure and certain cardiac myopathies, muscle weakness, hyperlipidaemias and atherosclerosis (Stacpoole et al., 1978; N. Engl. J. Med. 298: 526-530). A compound that activates PDH may also be useful in treating Alzheimer's disease (AD) (J Neural Transm (1998) 105, 855-870).

European Patent Publication Nos. 617010 and 524781 describe compounds which are capable of relaxing bladder smooth muscle and which may be used in the treatment of urge incontinence. International Applications WO 9944618, WO 9947508, WO 9962506, WO 9962873, WO 01/17942, WO 01/17955 and WO 01/17956 describe compounds that elevate PDH activity. The compounds of the present invention are not specifically disclosed in any of the above applications and we have surprisingly found that these compound possess beneficial properties in terms of one or more of their pharmacological activity (particularly as compounds which elevate pyruvate dehydrogenase) and/or pharmacokinetic, efficacious, metabolic and toxicological profiles that make them particularly suitable for in vivo administration to a warm blooded animal, such as man.

Accordingly the present invention provides a compound of formula (I): 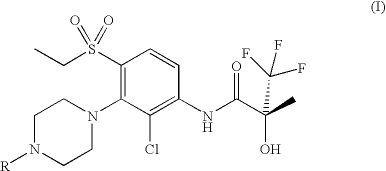
wherein R is methyl or mesyl;
or a pharmaceutically acceptable salt or an in vivo hydrolysable ester thereof.

In one aspect of the invention R is methyl.

In a further aspect of the invention R is mesyl.

Further aspects of the invention are those which relate to a compound or a pharmaceutically acceptable salt thereof.

It is also to be understood that a compound of formula (I) and its pharmaceutically acceptable salts and in vivo hydrolysable esters thereof can exist in solvated as well as unsolvated forms such as, for example, hydrated forms. It is to be understood that the invention encompasses all such solvated forms which elevate PDH activity.

A compound of formula (I) and its pharmaceutically acceptable salts and in vivo hydrolysable esters thereof may be prepared by any process known to be applicable to the preparation of chemically related compounds. Such processes include, for example, those illustrated in European Patent Applications, Publication Nos. 0524781, 0617010, 0625516, and in GB 2278054 and in International Applications WO 9323358, WO 9738124, WO 9944618, WO 9947508, WO 9962506, WO 9962873, WO01/17942, WO 01/17955 and WO01/17956.

Another aspect of the present invention provides a process for preparing compounds of formula (I) or a pharmaceutically acceptable salt or an in vivo hydrolysable ester thereof, which process (in which variable groups are as defined for formula ( ) unless otherwise stated) comprises of:
(a) deprotecting a protected compound of formula (II): 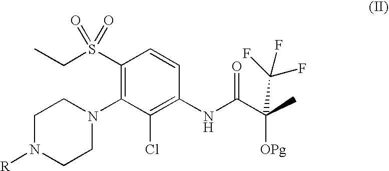
   where Pg is an alcohol protecting group;
(b) oxidising a compound of formula (III): 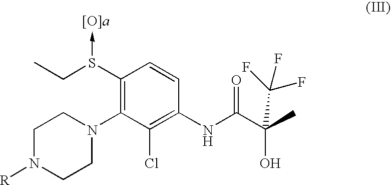
   wherein a is 0 or 1;
(c) coupling a compound of formula (IV): 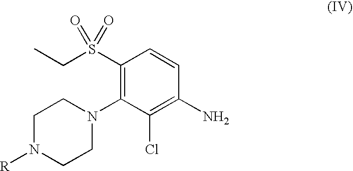
   with the acid of formula (V): 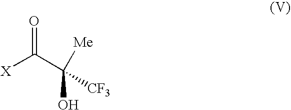
   wherein X is OH;
(d) coupling an aniline of formula (IV) with an activated acid derivative of formula (V);
(e) reacting a compound of formula (VI): 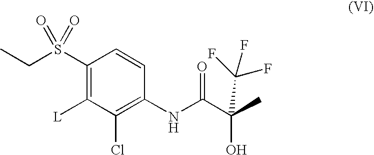
   wherein L is a displaceable group; with 4-mesylpiperazine or 4-methylpiperazine;
(f) for compounds of formula (I) wherein R is methyl; methylating the compound of formula (VII): 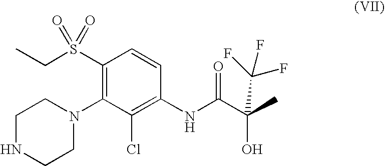
(g) for compounds of formula (I) wherein R is mesyl; mesylating the compound of formula (VII);
(b) chlorination of a compound of formula (VIII): 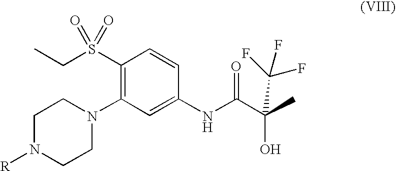
(i) functional group conversion to chlorine of a compound of formula (IX): 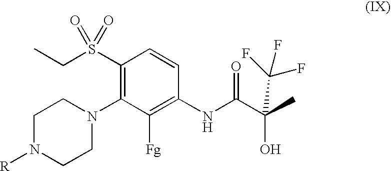
   wherein Fg is a functional group;
(j) addition of an organometallic reagent to a compound of formula (X): 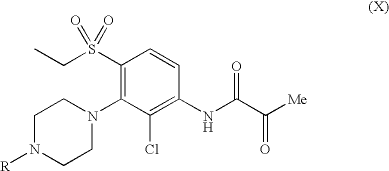
(k) addition of an organometallic reagent to a compound of formula (XI): 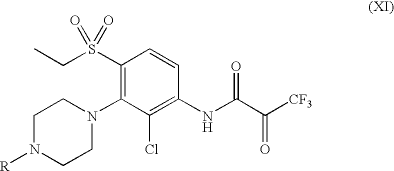
(l) addition of a compound of formula (V) wherein X is NH₂ to a compound of formula (XII): 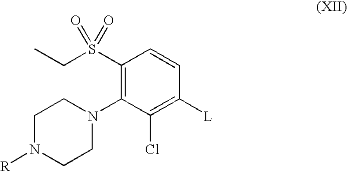
   wherein L is a displaceable group;
(m) Smiles rearrangement of a compound of formula (XIII): 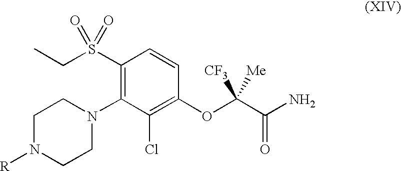
   or
(n) separating a mixture of the (R) and (S) enantiomers of compounds of formula (I) to give the (R)-enantiomer;
   and thereafter if required forming a pharmaceutically acceptable salt or in vivo hydrolysable ester.

Suitable values for Pg are a benzyl group, a silyl group (for example a trialkylsilyl group or an alkyldiphenylsilyl group) or an acetyl protecting group.

Where formula (V) is an activated acid derivative, suitable values for X include halo (for example chloro or bromo), anhydrides, aryloxys (for example 4-nitrophenoxy or pentafluorophenoxy) or imidazol-1-yl.

L is a displaceable group. Suitable values for L include fluoro, chloro, bromo, nitro, methanesulphonate and trifluoromethanesulphonate.

Fg is a functional group. A suitable functional group is amino which could be interconverted by diazotisation and reaction of the diazonium salt with chloride under copper catalysis.

Specific conditions of the above reactions are as follows:

Process (a)

Examples of suitable reagents for deprotecting an alcohol of formula (II) are:
1) when Pg is benzyl:
(i) hydrogen in the presence of palladium/carbon catalyst, i.e. hydrogenolysis; or
(ii) hydrogen bromide or hydrogen iodide;
2) when Pg is a silyl protecting group:
(i) tetrabutylammonium fluoride; or
(ii) hydrofluoric or hydrochloric acid;
3) when Pg is acetyl:
i) mild aqueous base for example lithium hydroxide; or
ii) ammonia or an amine such as dimethylamine.

The reaction can be conducted in a suitable solvent such as ethanol, methanol, acetonitrile, or dimethylsulphoxide and may conveniently be performed at a temperature in the range of −40 to 100° C.

Compounds of formula (II) may be prepared according to the following scheme:

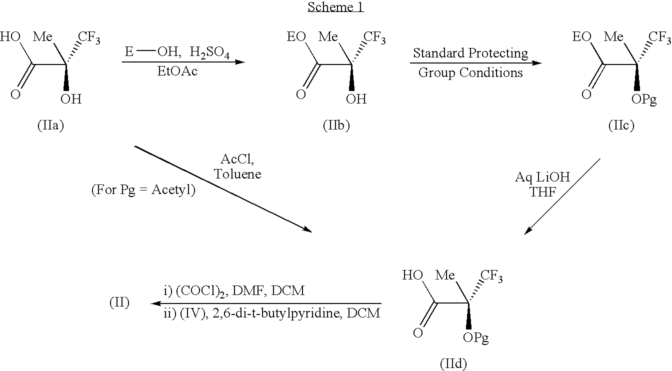

E is a carboxy protecting group. Suitable values for E include C₁₋₆alkyl, such as methyl and ethyl.

The compound of formula (Ha) is a commercially available compound.

Process (b)

Suitable oxidising agents include potassium permanganate, OXONE™, sodium periodate, peracids (such as for example 3-chloroperoxybenzoic acid or peracetic acid), hydrogen peroxide, TPAP (tetrapropylammonium perruthenate) or oxygen. The reaction may be conducted in a suitable solvent such as diethyl ether, DCM, methanol, ethanol, water, acetic acid, or mixtures of two or more of these solvents. The reaction may conveniently be performed at a temperature in the range of −40 to 100° C.

Compounds of formula (III) may be prepared according to the following scheme:

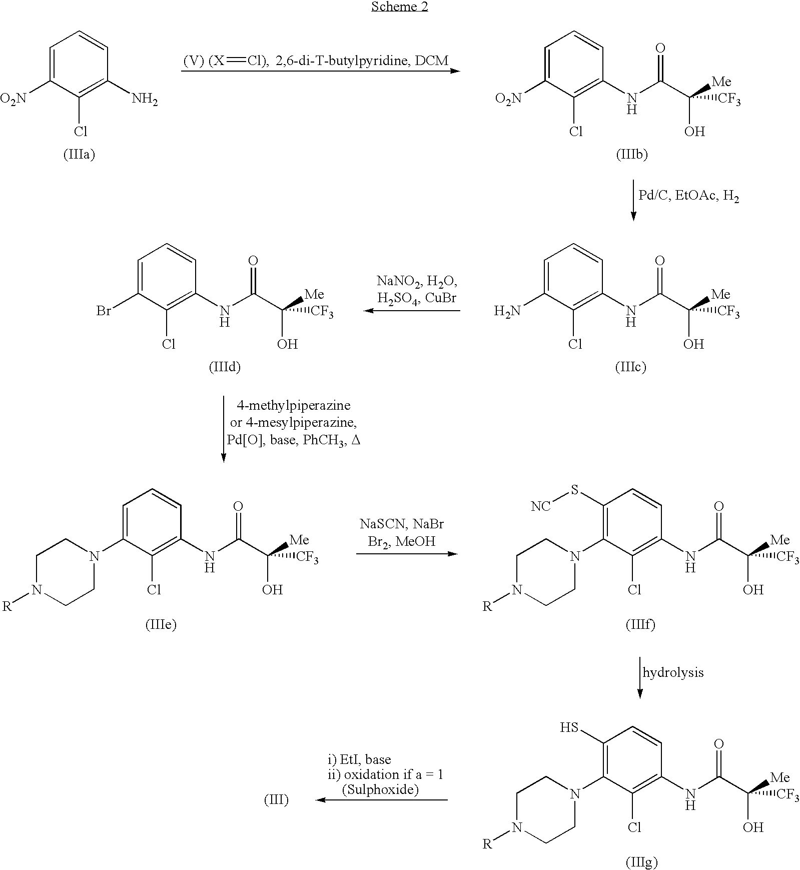

The compound of formula (IIIa) is a commercially available compound.

Process (c)

The reaction can be conducted in the presence of a suitable coupling reagent. Standard amide coupling reagents known in the art can be employed as suitable coupling reagents, for example conditions such as those described above for the coupling of (IIIa) and (V) or (IV) and (IId), or for example dicyclohexyl-carbodiimide, optionally in the presence of a catalyst such as dimethylaminopyridine or 4-pyrrolidinopyridine, optionally in the presence of a base for example triethylamine, pyridine, or 2,6-di-alkyl-pyridines (such as 2,6-lutidine or 2,6-di-tert-butylpyridine) or 2,6-diphenylpyridine. Suitable solvents include dimethylacetamide, DCM, benzene, THF and DMF. The coupling reaction may conveniently be performed at a temperature in the range of −40 to 40° C.

Compounds of formula (IV) maybe prepared according to the following scheme:

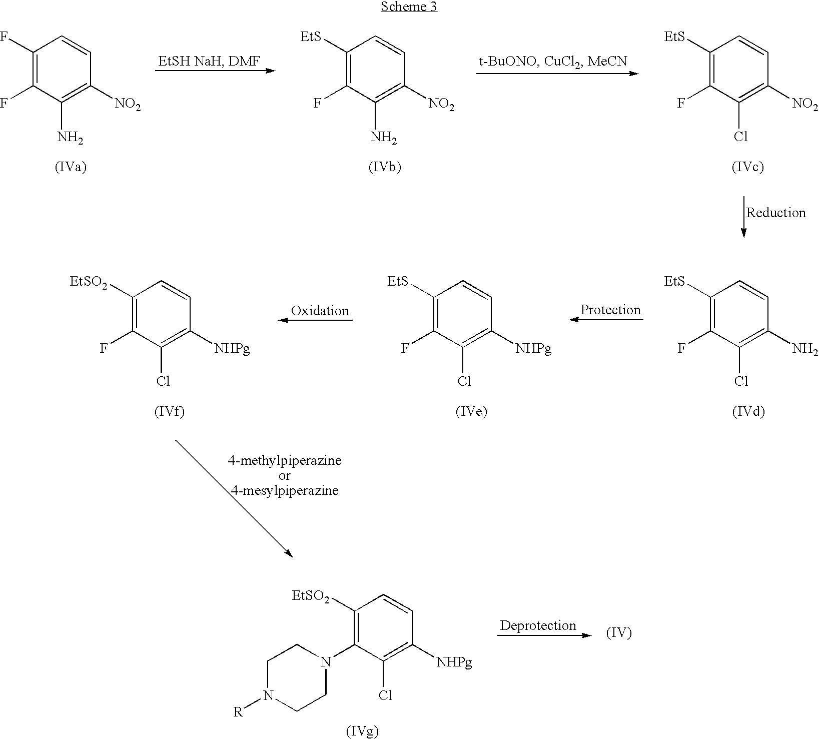

Pg is an amine protecting group such as those described below.

The compounds of formula (IVa) and (V) are commercially available compounds and they are known in the literature.

For example, the resolved acid of formula (V) maybe prepared by any of the known methods for preparation of optically-active forms (for example, by recrystallization of the chiral salt {for example WO 9738124}, by enzymatic resolution or by chromatographic separation using a chiral stationary phase). For example the (R)-(+) resolved acid may be prepared by the method of Scheme 2 in World Patent Application Publication No. WO 9738124 for preparation of the (S)-(−) acid, i.e. using the classical resolution method described in European Patent Application Publication No. EP 0524781, also for preparation of the (S)-(−) acid, except that (1S,2R)-norephedrine is used in place of (S)-(−)-1-phenylethylamine. The chiral acid may also be prepared by using the enzymatic resolution method as described in Tetrahedron Asymmetry, 1999, 10, 679.

Process (d)

This coupling may be achieved optionally in the presence of a base for example triethylamine, pyridine, 2,6-di-allyl-pyridines (such as 2,6-lutidine or 2,6-di-tert-butylpyridine) or 2,6-diphenylpyridine. Suitable solvents include dimethylacetamide, DCM, benzene, TIF and DMF. The coupling reaction may conveniently be performed at a temperature in the range of −40 to 40° C.

Process (e)

This reaction may be achieved by reaction of 1-mesylpiperazine (U.S. Pat. No. 6,140,351) or 1-methylpiperazine (1-20 molar equivalents, preferably 2-10 equivalents) with (VI) in a solvent such as N-methyl-2-pyrrolidinone or dimethylacetamide, or neat, with heating at a temperature of from 40 to 160° C.

Compounds of formula (VI) wherein L is fluoro may be prepared by the following scheme. 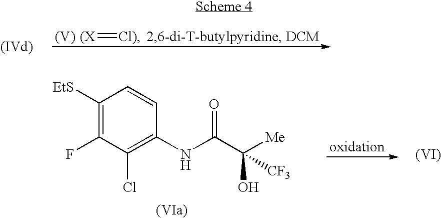
Process (f)

Compound (VII) may be methylated using formaldehyde and a reducing agent such as sodium borohydride or sodium triacetoxyborohydride in a suitable solvent such as 1,2-dichloroethane, DCM or THF, at a temperature in the range of 0° C. to reflux, preferably at or near room temperature. Alternatively compound (VII) may be methylated using a methylating agent such as methyl iodide or dimethylsulphate in a solvent such as acetone or DMF in the presence of a base such as sodium bicarbonate, sodium carbonate or sodium hydroxide, optionally with protection of the hydroxy group. A preparation of Compound (VII) is described under Method 1 below.

Process (g)

Compound (VII) may be mesylated using a suitable agent such as methanesulphonyl chloride, in the presence of a base, such as triethylamine, in a suitable solvent such as DCM, TEF, pyridine or ethyl acetate, at a temperature in the range of −40° C. to reflux, preferably at or near room temperature.

Process (h)

The chlorination may be carried out for example using N-chlorosuccinimide in a solvent such as DCM, acetonitrile, isopropanol or DMF at a temperature in the range of 0° C. to reflux, or using chlorine in the presence of a catalyst such as iron trichloride in a suitable solvent such as acetic acid, DMF or acetonitrile, at a temperature in the range of −20° C. to 40° C., preferably at or below room temperature, followed by separation of the required product from unwanted isomeric inpurities, if formed.

Compounds of formula (VIII) may be prepared according to the following scheme:

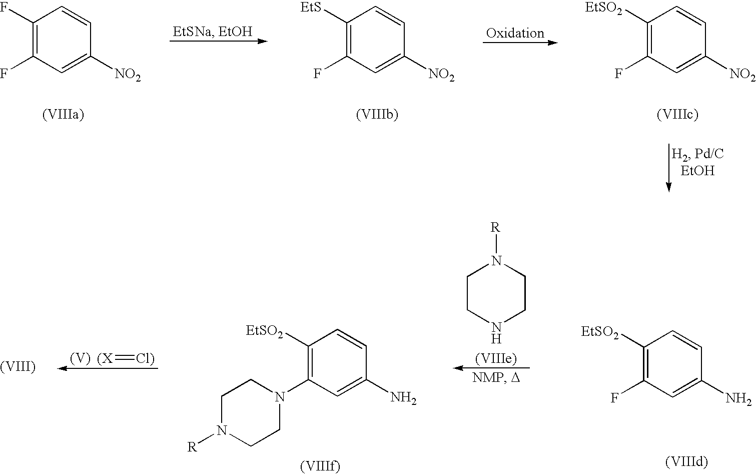

Compound (VIIIa) is commercially available.

Process (i)

These functional group interconversions use reagents and reaction conditions well known in the chemical art.

For example, the functional group interconversion of (IX) wherein Fg is NH₂ into the a compound of formula (I) may be carried out by diazotisation for example with t-butylnitrite etc in the presence of a catalyst such as cupric chloride in a solvent such as acetonitrile, at a temperature in the range of 0° C. to reflux, preferably at or near room temperature. Alternatively the conversion may be carried out by diazotisation with a nitrite salt in the presence of an acid such as HCl or sulphuric acid in a solvent such as water, acetic acid or mixtures of the two, at a temperature of from −20 to 40° C., followed by reaction of the thus-formed diazonium salt with cuprous chloride in the same solvent at a temperature of from 0° C. to reflux.

Compounds of formula (IX) may be prepared according to the following scheme: 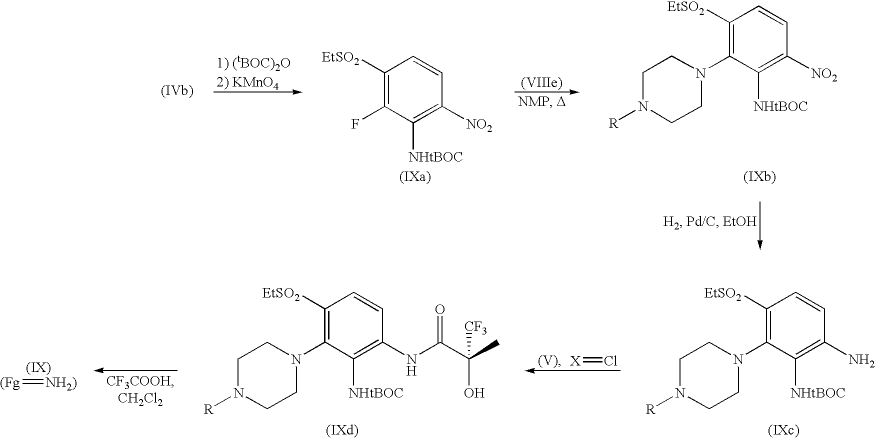
Process (i)

The reaction can be carried out by the addition of a suitable reagent such as CF₃SiMe₃ (Ruppert's reagent, Tetrahedron, 2000, 56(39), 7613), which may be achieved asymmetrically using a suitable catalyst such as a chiral cinchoninium fluoride catalyst, (Tetrahedron Lett., 1994, 35, 3137), and subsequent acidic aqueous work-up to effect hydrolysis of the tertiary alcohol silyl ether generated in the reaction. This reaction may be carried out in a suitable solvent such as toluene, at a temperature in the range of −100° C. to room temperature to reflux, preferably at −78° C. to room temperature.

Compounds of formula (X) may be prepared by the method of process (c), i.e. by coupling a compound (MV) with pyruvic acid instead of an acid of formula (V).

Process (k)

The reaction can be carried out by the addition of a suitable organometallic reagent such as CH₃MgBr or CH₃CeCl₂ in a solvent such as ether or THF at a temperature of −120 to 40° C. in the presence of a chiral catalyst such as a TADDOL (Tetraaryldimethyldioxolane-dimethanol, for example where aryl is phenyl or 2-naphthyl; Angew. Chem. Int. Ed. Engl., 1992, 31, 84-6).

Compounds of formula (XI) may be prepared by the method of process (c), i.e. by coupling a compound (IV) with trifluoropyruvic acid (Tetrahedron Lett., 1989, 30(39), 5243) instead of an acid of formula (V).

Process (l)

The reaction can be carried out by reacting a compound of formula (XII) with the dianion formed by treating the compound of formula (V) wherein X is NH₂ with two molar equivalents of base such as sodium hydride in a suitable solvent such as TBF or NMP, at a temperature of from 20-160° C.

A compound of formula (XII) wherein L is Cl may be prepared for example by diazotisation of a compound of formula (IV) using the process as described in Process (i).

Process (m)

The Smiles rearrangement can be carried out by treatment of a compound of formula (XIV) with a base such as sodium hydride in a solvent such as DMF.

A compound of formula (XIV) may be prepared from a compound of formula (XII) wherein L is Cl with a compound of formula (V) wherein X is NH₂ with one molar equivalent of base such as sodium hydride in a solvent such as THF or NMP, at a temperature of from 20-160° C.

Process (n)

The required optically active form of a compound of formula (I) may be obtained by resolution of a mixture of a compound of formula (I) and its corresponding (S) enantiomer using standard procedures well known to those skilled in the art, for example, crystallisation, enzymatic resolution or chromatographic separation of enantiomers.

If not commercially available, the necessary starting materials for the procedures such as those described above may be made by procedures which are selected from standard organic chemical techniques, techniques which are analogous to the synthesis of known, structurally similar compounds, or techniques which are analogous to the above described procedure or the procedures described in the examples.

It is noted that many of the starting materials for synthetic methods as described above are commercially available and/or widely reported in the scientific literature, or could be made from commercially available compounds using adaptations of processes reported in the scientific literature. The reader is further referred to Advanced Organic Chemistry, 4^{th} Edition, by Jerry March, published by John Wiley & Sons 1992, for general guidance on reaction conditions and reagents.

It will also be appreciated that in some of the reactions mentioned herein it may be necessary/desirable to protect any sensitive groups in compounds. The instances where protection is necessary or desirable are known to those skilled in the art, as are suitable methods for such protection. Conventional protecting groups may be used in accordance with standard practice (for illustration see T. W. Greene, Protective Groups in Organic Synthesis, John Wiley and Sons, 1991).

Examples of a suitable protecting group for a hydroxy group is, for example, an acyl group, for example an alkanoyl group such as acetyl, an aroyl group, for example benzoyl, a silyl group such as trimethylsilyl or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively a silyl group such as trimethylsilyl may be removed, for example, by fluoride or by aqueous acid; or an arylmethyl group such as a benzyl group may be removed, for example, by hydrogenation in the presence of a catalyst such as palladium-on-carbon.

A suitable protecting group for an amino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifiuoroacetate). A suitable alternative protecting group for a primary amino group is, for example, a phthaloyl group which may be removed by treatment with an alkylamine, for example dimethylaminopropylamine or 2-hydroxyethylamine, or with hydrazine.

The protecting groups may be removed at any convenient stage in the synthesis using conventional techniques well known in the chemical art, or they may be removed during a later reaction step or work-up.

A compound of formula (I) may form stable acid or basic salts, and in such cases administration of a compound as a salt may be appropriate, and pharmaceutically acceptable salts may be made by conventional methods such as those described following. Examples of suitable pharmaceutically acceptable salts are organic acid addition salts formed with acids which form a physiologically acceptable anion, for example, tosylate, methanesulphonate and α-glycerophosphate. Suitable inorganic salts may also be formed such as sulphate, nitrate, and chloride.

Pharmaceutically acceptable salts may be obtained using standard procedures well known in the art, for example by reacting a compound of formula (I) (and in some cases the ester) with a suitable acid affording a physiologically acceptable anion. It is also possible to make a corresponding alkali metal (e.g. sodium, potassium, or lithium) or alkaline earth metal (e.g. calcium) salt by treating a compound of formula (I) (and in some cases the ester) with one equivalent of an alkali metal hydroxide or alkoxide or half an equivalent of alkaline earth metal hydroxide or alkoxide (e.g. the ethoxide or methoxide) in aqueous medium followed by conventional purification techniques.

An in vivo hydrolysable ester of a compound of formula (I) is, for example, a pharmaceutically acceptable ester which is hydrolysed in the human or animal body to produce the parent acid or alcohol.

Suitable in vivo hydrolysable esters of a compound of formula (I) formed with the hydroxy group includes inorganic esters such as phosphate esters and α-acyloxyalkyl ethers. Examples of α-acyloxyalkyl ethers include acetoxymethoxy and 2,2-dimethylpropionyloxy-methoxy. Other in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. Examples of substituents for benzoyl include morpholino and piperazino linked from a ring nitrogen atom via a methylene group to the 3- or 4 position of the benzoyl ring.

The identification of compounds that elevate PDH activity is the subject of the present invention. These properties may be assessed, for example, using one or more of the test procedures known in the literature, for example those set out in WO 9962506; namely test (a)—in vitro elevation of PDH activity, test (b)—in vitro elevation of PDH activity in isolated primary cells and test (c) in vivo elevation of PDH activity and these tests are incorporated herein by reference. Alternatively these properties may be assessed in the following test:

In Vitro Elevation of PDH Activity

This assay determines the ability of a test compound to elevate PDH activity cDNA encoding PDH kinase may be obtained by Polymerase Chain Reaction (PCR) and subsequent cloning. This may be expressed in a suitable expression system to obtain polypeptide with PDH kinase activity. For example human PDHkinase2 (RPDIK2) obtained by expression of recombinant protein in Escherichia coli (E. Coli), was found to display PDH kinase activity.

Human rPDHK2 (Genbank accession number L42451.1) was cloned and expressed by the method described in Baker et. al. (2000) J. Biol. Chem. 275, 15773-15781. A protease cleavage site was incorporated into the expressed protein as described in this reference. Other known PDH kinases for use in assays, may be cloned and expressed in a similar manner. For expression of rPDB2 activity, E. coli strain BL21 (DE3) cells were transformed with the pET28A vector containing rPDHK2 cDNA. This vector incorporates a 6-His tag onto the protein at its N-terminus. E. coli were grown in a fermenter to an optical density of 12 (550 nm) at 37° C., reducing to 22° C. until an optical density of 15 was achieved and protein expression was induced by the addition of 0.5 mM isopropylthio-β-galactosidase. Cells were grown for 3 hours at 22° C. and harvested by centrifugation. The resuspended cell paste was lysed by high pressure homogenisation and insoluble material removed by centrifugation at 26000×g for 30 minutes. The 6-His tagged protein was removed from the supernatant using a cobalt chelating resin (TALON: Clontech) matrix which was washed in 20 mM N-[2-hydroxyethyl]piperazine-N′-[2-ethanesulphonic acid (HEPES), 500 mM NaCl, 1%(v/v) ethylene glycol, 0.1%(w/v) Pluronics F-68 pH8.0, prior to a progressive stepped elution of the bound protein using a similar buffer with the addition of 100 mM imidazole pH8.0. Eluted fractions containing the 6-His tagged protein were pooled, ethylene diaminetetracetic acid (EDTA) and dithiothreitol (DTT) were added to a final concentration of 1 mM and the tag cleaved by the addition of PreScission Protease (Amersham Pharmacia Biotech). This protease was removed using Glutathione Sepharose (Amersham Pharmacia Biotech). The untagged protein was dialysed into a storage buffer of 20 mM HEPES-Na, 150 mM sodium chloride, 0.5 mM EDTA, 1%(w/v) Pluronics F68, 1%(v/v) ethylene glycol pH8.0 and stored in aliquots at −80° C.

Each new batch of stock PDHK enzyme was titrated in the assay to determine a concentration giving approximately 75% inhibition of PDH in the conditions of the assay. Stock enzyme (typically 20 μg/ml) was allowed to associate for 24 hours at 4° C. with PDH (porcine heart PDH Sigma P7032) (0.05U/ml) in a buffer containing 50 mM 3-[N-Morpholino]propane sulphonic acid (MOPS), 20 mM dipotassium orthophosphate, 60 mM potassium chloride, 2 mM magnesium chloride, 0.4 mM ethylene diaminetetracetic acid (EDTA), 0.2% Pluronic F68, 1 mM dithiothreitol (DTM), pH7.3.

For assay of the activity of novel compounds, compounds were diluted in 5% DMSO and 5 μl transferred to individual wells of 384-well assay plates. Control wells contained 5 μl 5% DMSO instead of compound. In order to determine maximum rate of the PDH reaction a second series of control wells was included containing 5 μl of a known inhibitor at a final concentration in the kinase reaction of 10 μM.

40 μl pre-associated enzyme solution was added and the phosphorylation reaction initiated by the addition of 5 μl 10 μM ATP in the above buffer. After 45 minutes at room temperature, the residual activity of the PDH was determined by the addition of substrates (2.5 mM coenzyme A, 2.5 mM thiamine pyrophosphate (cocarboxylase), 2.5 mM sodium pyruvate, 6 mM NAD) in a volume of 40 μl and the plates were incubated for 90 minutes at ambient temperature. The production of reduced NAD (NADH) was established by measured optical density at 340 nm using a plate reading spectrophotometer. The EC₅₀ for a test compound was determined in the usual way using results from 12 concentrations of the compound.

According to a further aspect of the invention there is provided a pharmaceutical composition which comprises a compound of formula (I) as defined hereinbefore or a pharmaceutically acceptable salt or an in vivo hydrolysable ester thereof, in association with a pharmaceutically acceptable excipient or carrier.

The composition may be in a form suitable for oral administration, for example as a tablet or capsule, for parenteral injection (including intravenous, subcutaneous, intramuscular, intravascular or infusion) for example as a sterile solution, suspension or emulsion, for topical administration for example as an ointment or cream or for rectal administration for example as a suppository. In general the above compositions may be prepared in a conventional manner using conventional excipients.

The compositions of the present invention are advantageously presented in unit dosage form. A compound will normally be administered to a warm-blooded animal at a unit dose within the range 5-5000 mg per square metre body area of the animal, i.e. approximately 0.1-100 mg/kg. A unit dose in the range, for example, 1-100 mg/kg, preferably 1-50 mg/kg is envisaged and this normally provides a therapeutically-effective dose. A unit dose form such as a tablet or capsule will usually contain, for example 1-250 mg of active ingredient.

According to a further aspect of the present invention there is provided a compound of formula (I) or a pharmaceutically acceptable salt or an in vivo hydrolysable ester thereof as defined hereinbefore for use in a method of treatment of the human or animal body by therapy.

We have found that compounds of the present invention elevate PDH activity and are therefore of interest for their blood glucose-lowering effects.

A further feature of the present invention is a compound of formula (I) and pharmaceutically acceptable salts or in vivo hydrolysable esters thereof for use as a medicament.

Conveniently this is a compound of formula (I), or a pharmaceutically acceptable salt or an in vivo hydrolysable ester thereof, for use as a medicament for producing an elevation of PDH activity in a warm-blooded animal such as a human being.

Particularly this is a compound of formula (I), or a pharmaceutically acceptable salt or an in vivo hydrolysable ester thereof, for use as a medicament for treating diabetes mellitus in a warm-blooded animal such as a human being.

Particularly this is a compound of formula (I), or a pharmaceutically acceptable salt or an in vivo hydrolysable ester thereof, for use as a medicament for treating diabetes mellitus, peripheral vascular disease and myocardial ischaemia in a warm-blooded animal such as a human being.

Thus according to a further aspect of the invention there is provided the use of a compound of formula (I), or a pharmaceutically acceptable salt or an in vivo hydrolysable ester thereof in the manufacture of a medicament for use in the production of an elevation of PDH activity in a warm-blooded animal such as a human being.

Thus according to a further aspect of the invention there is provided the use of a compound of formula (I), or a pharmaceutically acceptable salt or an in vivo hydrolysable ester thereof in the manufacture of a medicament for use in the treatment of diabetes mellitus in a warm-blooded animal such as a human being.

Thus according to a further aspect of the invention there is provided the use of a compound of formula (I), or a pharmaceutically acceptable salt or an in vivo hydrolysable ester thereof in the manufacture of a medicament for use in the treatment of diabetes mellitus, peripheral vascular disease and myocardial ischaemia in a warm-blooded animal such as a human being.

According to a further aspect of the invention there is provided a pharmaceutical composition which comprises a compound of formula (I) as defined hereinbefore or a pharmaceutically acceptable salt or an in vivo hydrolysable ester thereof, in association with a pharmaceutically acceptable excipient or carrier for use in producing an elevation of PDH activity in an warm-blooded animal, such as a human being.

According to a further aspect of the invention there is provided a pharmaceutical composition which comprises a compound of formula (I) as defined hereinbefore or a pharmaceutically acceptable salt or an in vivo hydrolysable ester thereof, in association with a pharmaceutically acceptable excipient or carrier for use in the treatment of diabetes mellitus in an warm-blooded animal, such as a human being.

According to a further aspect of the invention there is provided a pharmaceutical composition which comprises a compound of formula (I) as defined hereinbefore or a pharmaceutically acceptable salt or an in vivo hydrolysable ester thereof in association with a pharmaceutically acceptable excipient or carrier for use in the treatment of diabetes mellitus, peripheral vascular disease and myocardial ischaemia in an warm-blooded animal, such as a human being.

According to a further feature of the invention there is provided a method for producing an elevation of PDH activity in a warm-blooded animal, such as a human being, in need of such treatment which comprises administering to said animal an effective amount of a compound of formula (I) or a pharmaceutically acceptable salt or an in vivo hydrolysable ester thereof as defined hereinbefore.

According to a further feature of the invention there is provided a method of treating diabetes mellitus in a warm-blooded animal, such as a human being, in need of such treatment which comprises administering to said animal an effective amount of a compound of formula (I) or a pharmaceutically acceptable salt or an in vivo hydrolysable ester thereof as defined hereinbefore.

According to a further feature of the invention there is provided a method of treating diabetes mellitus, peripheral vascular disease and myocardial ischaemia in a warm-blooded animal, such as a human being, in need of such treatment which comprises administering to said animal an effective amount of a compound of formula (I) or a pharmaceutically acceptable salt or an in vivo hydrolysable ester thereof as defined hereinbefore.

As stated above the size of the dose required for the therapeutic or prophylactic treatment of a particular disease state will necessarily be varied depending on the host treated, the route of administration and the severity of the illness being treated. Preferably a daily dose in the range of 1-50 mg/kg is employed. However the daily dose will necessarily be varied depending upon the host treated, the particular route of administration, and the severity of the illness being treated. Accordingly the optimum dosage may be determined by the practitioner who is treating any particular patient.

As stated above compounds defined in the present invention are of interest for their ability to elevate the activity of PDH. A compound of the invention may therefore be useful in a range of disease states including diabetes mellitus, peripheral vascular disease, (including intermittent claudication), cardiac failure and certain cardiac myopathies, myocardial ischaemia, cerebral ischaemia and reperfusion, muscle weakness, hyperlipidaemias, Alzheimer's disease and/or atherosclerosis. Alternatively such compounds of the invention may be useful in a range of disease states including peripheral vascular disease, (including intermittent claudication), cardiac failure and certain cardiac myopathies, myocardial ischaemia, cerebral ischaemia and reperfusion, muscle weakness, hyperlipidaemias, Alzheimer's disease and/or atherosclerosis in particular peripheral vascular disease and myocardial ischaemia.

In addition to its use in therapeutic medicine, compounds of formula (I) and their pharmaceutically acceptable salts and in vivo hydrolysable esters are also useful as pharmacological tools in the development and standardisation of in vitro and in vivo test systems for the evaluation of the effects of elevators of PDH activity in laboratory animals such as cats, dogs, rabbits, monkeys, rats and mice, as part of the search for new therapeutic agents.

The invention will now be illustrated by the following non-limiting example in which, unless stated otherwise:
(i) temperatures are given in degrees Celsius (° C.); operations were carried out at room or ambient temperature, that is, at a temperature in the range of 18-25° C. and under an atmosphere of an inert gas such as argon;
(ii) organic solutions were dried over anhydrous magnesium sulphate; evaporation of solvent was carried out using a rotary evaporator under reduced pressure (600-4000 Pascals; 4.5-30 mmHg) with a bath temperature of up to 60° C.;
(ii) chromatography means flash chromatography on silica gel; where a Biotage cartridge is referred to this means a cartridge containing KP-SIL™ silica, 60 Å, particle size 32-63 mM, supplied by Biotage, a division of Dyax Corp., 1500 Avon Street Extended, Charlottesville, Va. 22902, USA;
(iv) in general, the course of reactions was followed by TLC and reaction times are given for illustration only;
(v) yields are given for illustration only and are not necessarily those which can be obtained by diligent process development; preparations were repeated if more material was required;
(vi) where given, NMR data is in the form of delta values for major diagnostic protons, given in parts per million (ppm) relative to tetramethylsilane (TMS) as an internal standard, determined at 300 MHz (unless otherwise stated) using perdeuterio dimethyl sulphoxide (DMSO-δ₆) as solvent; and peak multiplicities are shown as follows: s, singlet; d, doublet; dd, double doublet; t, triplet; it, triple triplet; q, quartet; tq, triple quartet; m, multiplet; br, broad;
(vii) chemical symbols have their usual meanings; SI units and symbols are used;
(viii) solvent ratios are given in volume:volume (v/v) terms;
(ix) mass spectra (MS) were run with an electron energy of 70 electron volts in the chemical ionisation (CI) mode using a direct exposure probe; where indicated ionisation was effected by electron impact (EI), fast atom bombardment (FAB) or electrospray (ESP); values for m/z are given; generally, only ions which indicate the parent mass are reported and unless otherwise stated the value quoted is (M-H)⁻;

(x) The following abbreviations are used:

| | |
|---|---|
| NMP | 1-methyl-2-pyrrolidinone; |
| DMF | N,N-dimethylformamide; |
| THF | tetrahydrofuran |
| DCM | dichloromethane; and |
| EtOAc | ethyl acetate; |

(xi) where (R) or (S) stereochemistry is quoted at the beginning of a name the indicated stereochemistry refers to the —NH—C(O)—C*(Me)(CF₃)(OH) centre as depicted in formula (I).

### EXAMPLE 1

(R)-N-[2-Chloro-4-ethylsulphonyl-3-(4-methylpiperazin-1-yl)phenyl]-2-hydroxy-2-methyl-3,3,3-trifluoropropanamide

Formaldehyde (0.77 g) and sodium triacetoxyborohydride (1.00 g) were added to a stirred solution of (R)-N-(2-chloro-4-ethylsulphonyl-3-piperazin-1-ylphenyl)-2-hydroxy-2-methyl-3,3,3-trifluoropropanamide (0.467 g; Method 1) in 1,2-dichloroethane (9 ml). The reaction mixture was stirred at an ambient temperature for 16 hours, then 1M NaOH solution (20 ml) was added, and the product was extracted with DCM (3×30 ml). Combined organic extracts were dried and volatile material was removed by evaporation. The residue was recrystallized from EtOAc/isohexane to give the title compound (0.315 g) as a solid. NMR: 1.11 (3H, t), 1.60 (3H, s), 2.10-2.18 (2H, m), 2.21 (3H, s), 2.70-2.82 (4H, m), 3.53 (2H, q), 3.55-3.62 (2H, m), 7.91 (1H, d), 8.07 (1H, brs), 8.23 (1H, d), 9.94 (1H, brs); m/z: 456.

### EXAMPLE 2

(R)-N-[2-Chloro-4-ethylsulphonyl-3-(4-methylpiperazin-1-yl)phenyl]-2-hydroxy-2-methyl-3,3,3-trifluoropropanamide (alternative preparation)

1-Methylpiperazine (0.102 g) was added to a stirred solution of (R)-N-(4-ethylsulphonyl-3-fluoro-2-chlorophenyl)-2-hydroxy-2-methyl-3,3,3-trifluoropropanamide (Example 15 of WO 01/17956; 0.096 g) in NMP (1 ml). The reaction mixture was heated at 130° C. for 24 hours. The reaction mixture was allowed to cool, then a saturated solution of ammonium chloride (100 ml) was added. The product was extracted with diethyl ether (3×100 ml). The organic extracts were dried, and volatile material was removed by evaporation. The residue was purified by chromatography on a Biotage cartridge (8 g silica) eluting with 5% methanol/DCM, to give the title compound (0.086 g) as a solid. NMR: 1.11 (3H, t), 1.60 (3H, s), 2.10-2.18 (2H, m), 2.21 (3H, s), 2.70-2.82 (4H, m), 3.53 (2H, q), 3.55-3.62 (2H, m), 7.91 (1H, d), 8.07 (1H, brs), 8.23 (1H, d), 9.94 (1H, brs); m/z: 456.

### EXAMPLE 3

(R)-N-[2-Chloro-4-ethylsulphonyl-3-(4-mesylpiperazin-1-yl)phenyl]-2-hydroxy-2-methyl-3,3,3-trifluoropropanamide

Triethylamine (0.091 g) and methanesulphonyl chloride (0.124 g) were added to a stirred suspension of (R)-N-(2-chloro-4-ethylsulphonyl-3-piperazin-1-ylphenyl)-2-hydroxy-2-methyl-3,3,3-trifluoropropanamide (0.401 g; Method 1) in DCM (10 ml). The reaction mixture was stirred at ambient temperature for 2 hours, then a saturated solution of ammonium chloride (20 ml) was added, and the product was extracted with DCM (3×30 ml). The organic extracts were dried and volatile material was removed by evaporation. The residue was purified by chromatography on a Biotage cartridge (8 g silica) eluting with 50-70% EtOAc/isohexane, to give the title compound (0.215 g) as a solid. NMR (CDCl₃): 1.26 (3H, t), 1.78 (3H, s), 2.86 (3H, s), 3.01-3.18 (4H, m), 3.39 (2H, q), 3.68 (1H, s), 3.75-3.87 (4H, m), 8.01 (1H, d), 8.57 (1H, d), 9.62 (1H, brs); m/z: 520.

### EXAMPLE 4

(R)-N-[2-Chloro-4-ethylsulphonyl-3-(4-mesylpiperazin-1-yl)phenyl]-2-hydroxy-2-methyl 3,3,3-trifluoropropanamide (alternative preparation)

1-Methanesulphonylpiperazine (0.370 g) was added to a stirred solution of (R)-N-(4-ethylsulphonyl-3-fluoro-2-chlorophenyl)-2-hydroxy-2-methyl-3,3,3-trifluoropropanamide (Example 15 of WO 01/17956; 0.213 g) in NMP (2 ml). The reaction mixture was heated at 150° C. for 48 hours, allowed to cool, then a saturated solution of ammonium chloride (100 ml) was added. The product was extracted with diethyl ether (3×100 ml). The organic extracts were dried and volatile material was removed by evaporation. The residue was purified by chromatography on a Biotage cartridge (8 g silica) eluting with 50-70% EtOAc/isohexane. The product was then recrystallized from EtOAc/isohexane to give the title compound (0.167 g) as a solid. NMR (CDCl₃): 1.26 (3H, t), 1.78 (3H, s), 2.86 (3H, s), 3.01-3.18 (4H, m), 3.39 (2H, q), 3.68 (1H, s), 3.75-3.87 (4H, m), 8.01 (1H, d), 8.57 (1H, d), 9.62 (1H, brs); m/z: 520.

Starting Material

Method 1

(R)—N-(2-Chloro-4-ethylsulphonyl-3-piperazin-1-ylphenyl)-2-hydroxy-2-methyl-3,3,3-trifluoropropanamide

t-Butyl 1-piperazinecarboxylate (6.12 g) was added to a stirred solution of R)—N-(4-ethylsulphonyl-3-fluoro-2-chlorophenyl)-2-hydroxy-2-methyl-3,3,3-trifluoropropanamide (Example 15 of WO 01/17956; 4.14 g) in NMP (15 ml). The reaction mixture was heated at 150° C. for 24 hours, allowed to cool, then a saturated solution of ammonium chloride (300 ml) was added. The product was extracted with diethyl ether (3×300 ml). The organic extracts were dried, and volatile material was removed by evaporation. The residue was purified by chromatography on a Biotage cartridge (90 g silica) eluting with 70% EtOAc/isohexane. The product was dissolved in trifluoroacetic acid (12 ml), then stirred at ambient temperature for 30 minutes. The reaction mixture was diluted with EtOAc (200 ml), then washed with 1M NaOH solution (300 ml). The organic extracts were dried and volatile material was removed by evaporation to give the title compound (3.52 g) as a solid. NMR: 1.12 (3H, t), 1.60 (3H, s), 2.74-2.86 (6H, m), 3.48-3.59 (4H, m), 7.89 (1H, d), 8.22 (1H, d); m/z: 442.

1. A compound of formula (I): 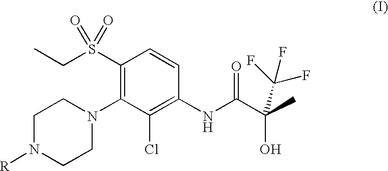
wherein R is methyl or mesyl;
or a pharmaceutically acceptable salt or an in vivo hydrolysable ester thereof.

2. A compound of formula (I) according to claim 1 wherein R is methyl or a pharmaceutically acceptable salt or an in vivo hydrolysable ester thereof.

3. A compound of formula (I) according to claim 1 wherein R is mesyl or a pharmaceutically acceptable salt or an in vivo hydrolysable ester thereof.

4. A process for preparing a compound of formula (I) or a pharmaceutically acceptable salt or an in vivo hydrolysable ester thereof as claimed in anyone of claims 1-3, which process (wherein R is as defined for formula (I) unless otherwise stated) comprises of:
(a) deprotecting a protected compound of formula (II): 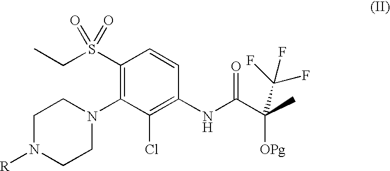
where pg is an alcohol protecting group;
(b) oxidising a compound of formula (III): 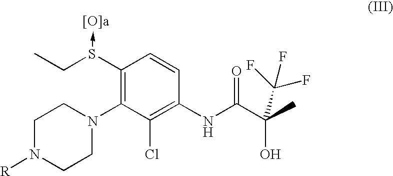
wherein a is 0 or 1;
(c) coupling a compound of formula (IV): 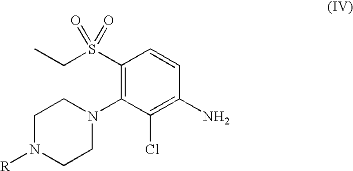
with the acid of formula (V): 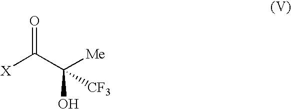
wherein X is OH;
(d) coupling an aniline of formula (IV) with an activated acid derivative of formula (V);
(e) reacting a compound of formula (VI): 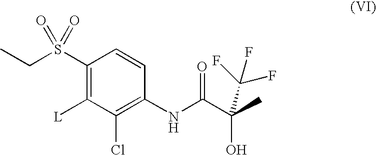
wherein L is a displaceable group; with 4-mesylpiperazine or 4-methylpiperazine;
(f) for compounds of formula (I) wherein R is methyl; methylating the compound of formula (VII): 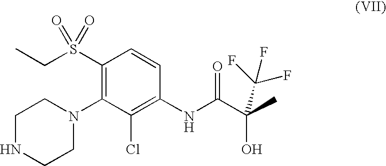
(g) for compounds of formula (l) wherein R is mesyl; mesylating the compound of formula (VII);
(h) chlorination of a compound of formula (VIII): 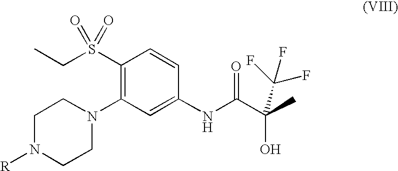
(i) functional group conversion to chlorine of a compound of formula (IX): 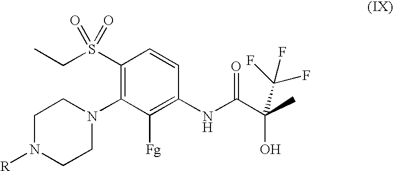
wherein Fg is a functional group;
(j) addition of an organometallic reagent to a compound of formula (X): 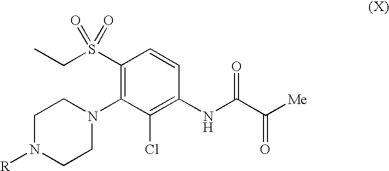
(k) addition of an organometallic reagent to a compound of formula (XI): 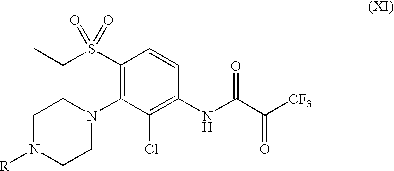
(l) addition of a compound of formula (V) wherein X is NH² to a compound of formula (XII): 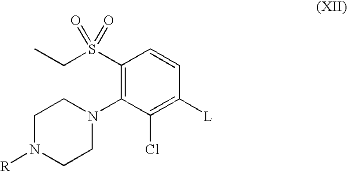
wherein L is a displaceable group;
(m) Smiles rearrangement of a compound of formula (XIII): 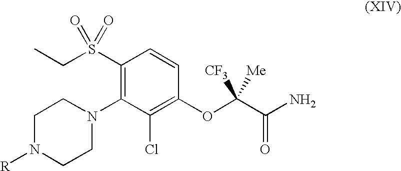
or
(n) separating a mixture of the (R) and (S) enantiomers of compounds of formula (I) to give the (R)-enantiomer;
and thereafter if required forming a pharmaceutically acceptable salt or in vivo hydrolysable ester.

5. A pharmaceutical composition which comprises a compound of formula (I) or a pharmaceutically acceptable salt or an in vivo hydrolysable ester thereof as claimed in anyone of claims 1-3, in association with a pharmaceutically acceptable excipient or carrier.

6. A compound of formula (I) or a pharmaceutically acceptable salt or an in vivo hydrolysable ester thereof as claimed in anyone of claims 1-3, for use as a medicament.

7. The use of a compound of formula (I), or a pharmaceutically acceptable salt or an in vivo hydrolysable ester thereof as claimed in anyone of claims 1-3, in the manufacture of a medicament for use in the production of an elevation of PDH activity in a warm-blooded animal such as a human being.

8. The use of a compound of formula (I), or a pharmaceutically acceptable salt or an in vivo hydrolysable ester thereof as claimed in anyone of claims 1-3, in the manufacture of a medicament for use in the treatment of diabetes mellitus in a warm-blooded animal such as a human being.

9. A method for producing an elevation of PDH activity in a warm-blooded animal, in need of such treatment which comprises administering to said animal an effective amount of a compound of formula (I) or a pharmaceutically acceptable salt or an in vivo hydrolysable ester thereof as claimed in anyone of claims 1-3.

10. A method of treating diabetes mellitus in a warm-blooded animal, such as a human being, in need of such treatment which comprises administering to said animal an effective amount of a compound of formula (I) or a pharmaceutically acceptable salt or an in vivo hydrolysable ester thereof as claimed in anyone of claims 1-3.

11. A pharmaceutical composition which comprises a compound of formula (I) or a pharmaceutically acceptable salt or an in vivo hydrolysable ester thereof as claimed in anyone of claims 1-3, in association with a pharmaceutically acceptable excipient or carrier for use in producing an elevation of PDH activity in an warm-blooded animal, such as a human being.

12. A pharmaceutical composition which comprises a compound of formula (I) or a pharmaceutically acceptable salt or an in vivo hydrolysable ester thereof as claimed in anyone of claims 1-3, in association with a pharmaceutically acceptable excipient or carrier for use in the treatment of diabetes mellitus in an warm-blooded animal, such as a human being.
